(19) United States
(12) Patent Application Publication
(10) Pub. No.: US 2023/0357472 A1
(43) Pub. Date: Nov. 9, 2023

(54) THERMOPLASTIC ELASTOMER MATERIALS WITH IMPROVED NON-SLIP PROPERTIES

(71) Applicant: Bixby International Corporation, Newburyport, MA (US)

(72) Inventors: Marc Gagnon, Exeter, NH (US); Ethel Bermejo, Somerville, MA (US); David P. Barkus, Springfield, IL (US); Chris Coco, Hampton, NH (US)

(21) Appl. No.: 18/313,237

(22) Filed: May 5, 2023

Related U.S. Application Data
(60) Provisional application No. 63/339,142, filed on May 6, 2022.

(51) Int. Cl. C08F 236/10; C08K 5/01

(52) U.S. Cl. CPC C08F 236/10; C08K 5/01; A61L 31/14

(57) ABSTRACT

A thermoplastic elastomer (TPE) material includes a styrene-ethylene-butadiene-styrene block copolymer rubber, a Food and Drug Administration (FDA) grade mineral oil, and a heat and UV stabilizer package. A method includes extruding a thermoplastic elastomer (TPE) material into a thin film roll with a thickness from 0.003″ to 0.010″, the TPE material comprising a styrene-ethylene-butadiene-styrene block copolymer rubber, a Food and Drug Administration (FDA) grade mineral oil, and a heat and UV stabilizer package.

### CROSS REFERENCE TO RELATED APPLICATIONS

This application claims benefit from U.S. Provisional Patent Application Ser. No. 63/339,142, filed May 6, 2022, which is incorporated by reference in its entirety.

### STATEMENT REGARDING GOVERNMENT INTEREST

Not applicable.

### BACKGROUND OF THE INVENTION

The present invention generally relates to polymeric materials, and in particular, to thermoplastic elastomer materials with improved no-slip properties.

Materials, such as heavy plasticized vinyl, may have non-slip properties that are useful in a variety of applications. Similarly, thermoset rubbers may also be utilized. However, these materials, among other similar materials with non-slip properties, also have other shortcomings.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In an aspect, the invention features a thermoplastic elastomer (TPE) material including a styrene-ethylene-butadiene-styrene block copolymer rubber, a Food and Drug Administration (FDA) grade mineral oil, and a heat and UV stabilizer package.

In another aspect, the invention features a method including extruding a thermoplastic elastomer (TPE) material into a thin film roll with a thickness from 0.003″ to 0.010″, the TPE material comprising a styrene-ethylene-butadiene-styrene block copolymer rubber, a Food and Drug Administration (FDA) grade mineral oil, and a heat and UV stabilizer package.

Embodiments may have one or more of the following advantages.

The TPE material can be processed on conventional equipment such as extrusion or injection molding. The non-slip material can be cast extruded into a thin film roll or to a sheet form and die-cut to any preferred geometry and size.

The TPE material can be recycled. After a die-cut process, any resultant waste can be collected, pelletized, and added back during the cast extrusion process up to 25% loadings as per industry standards.

The extruded TPE blend is soft, tear-resistant, and can withstand dishwasher or regular cleaning with soap and warm water. After cleaning, the material can recover its inherent grip properties even after dust accumulates on the surface over a period.

The non-slip TPE material can be cleaned with common medical disinfectants (e.g., hydrogen peroxide, IPA, and so forth) without dramatically changing the coefficient of friction.

The TPE material may be extruded or injection molded with a smooth or embossed texture. An embossed pattern can alter the material's tribological properties, further improving the grip properties of the non-slip material in wet conditions.

The TPE material is on-toxic, PVC and latex free.

The TPE material is easy to cut to any size or shape required.

The TPE material is dishwasher safe and reusable.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other aspects will now be described in detail with reference to the accompanying drawings, wherein:

FIG. 1 illustrates an exemplary SEBS tri-block copolymer ABA.

FIG. 2 illustrates exemplary components of a TPE mixture.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

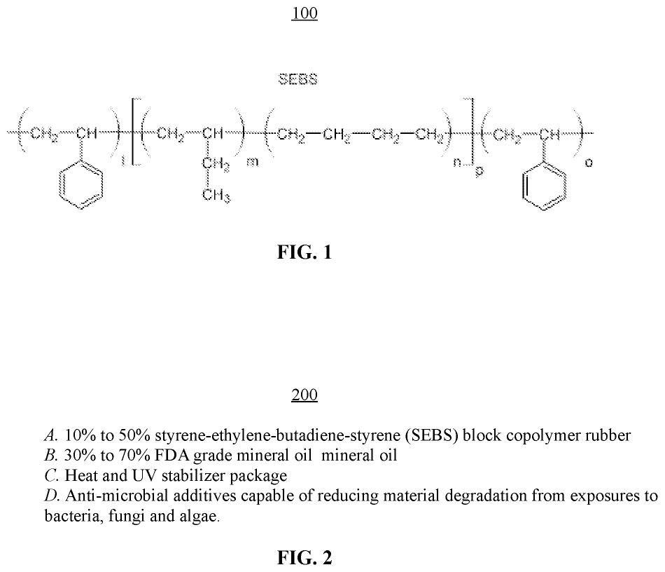

Disclosed herein are various embodiments of materials including thermoplastic elastomers (TPE). In some embodiments, the TPE include styrene-block copolymers, more specifically, styrene-ethylene-butadiene-styrene (SEBS) block copolymers. In FIG. 1, an example of a SEBS tri-block copolymer ABA (Acrylonitrile Butadiene Acrylate) is illustrated.

In other embodiments, the TPE including SEBS-block copolymers may include additional materials such as mineral oil and additives. The disclosed materials may have improved non-slip properties for various applications.

In some embodiments, thermoplastic resins may be clear. In other embodiments, the resins may be colored (e.g., blue or black). If necessary, additives and/or fillers may be added to affect the opaqueness of the resins. In some embodiments, printed layers may be included to create non-slip or high decor surfaces.

As shown in FIG. 2, in one embodiment, the TPE material 200 may include a mixture of the following components: (1) styrene-ethylene-butadiene-styrene (SEBS) block copolymer rubber ranging from about 10% to about 50% of the total composition of the TPE material; (2) mineral oil (e.g., FDA grade) ranging from about 30% to about 70% of the total composition of the TPE material; (3) heat and UV stabilizer package; and (4) anti-microbial additives that are capable of reducing material degradation from exposures to bacteria, fungi and algae.

In some embodiments, the TPE material may be processed with conventional equipment such as extrusion or injection molding. In some embodiments, the TPE material can be cast extruded into a thin film roll with thicknesses ranging from about 75 micrometers to about 250 micrometers, among other suitable thicknesses. In other embodiments, the TPE material can be cast extruded into sheet form (e.g., thickness of greater than about 250 micrometers) and die-cut to any geometric shapes or sizes. The TPE material can also be cast extruded into sheet form at different thicknesses.

In some embodiments, the TPE material (e.g., extruded) may have static coefficient of friction of at least greater than about 1.5 and a dynamic coefficient of friction of at least about 1.2.

In one embodiment, the presently disclosed TPE materials may be recycled or reused. After a die-cut process, any resulting waste may be collected, pelletized, and added back into a cast extrusion process. In some instances, the TPE material may be recycled up to a loading of about 25% per industry standard.

In one embodiment, the TPE material (e.g., extruded) may be soft, with a measured hardness of less than about 15A durometer according to the Shore A scale. The TPE material may also be tear-resistant and may be dishwasher safe or cleaning with soap and warm water.

After cleaning, the TPE material can recover its inherent non-slip properties even after dust has accumulated on the surface of the TPE material over a period of time. In contrast, heavy plasticized vinyl or thermoset rubbers may lose their intrinsic non-slip properties due to plasticizer migration or chemical reactions.

In some embodiments, the TPE material may be cleaned with medical-grade disinfectants (e.g., hydrogen peroxide, isopropyl alcohol) without adverse impact of its coefficient of friction.

In some embodiments, the TPE material may be extruded, or injection molded, among other forming techniques. In other embodiments, the TPE material may be formed with smooth or embossed textures, among other suitable textures.

In some instances, an embossed pattern may alter the material's tribological properties, further improving the gripping properties of the non-slip material in wet conditions or environments.

Currently disclosed embodiments may be used in a variety of applications. For example, in one embodiment, the disclosed TPE materials may be used as a liner to prevent objects from sliding off a flat surface, e.g., in boats, vehicles or food trays. In another embodiment, the disclosed TPE materials may be used as a grip material for tools, e.g., grip coating on handles. In some embodiments, the disclosed TPE materials may be used as casing or coating material for electronic devices such as laptops and mobile phones. In other embodiments, the disclosed TPE materials may be used as a floor-mat material, e.g., coatings or liners on floor mats.

In some embodiments, the presently disclosed non-slip TPE materials may be used in a variety of applications in the following industries: marine industry (e.g., boat planks), consumer goods industry (e.g., baby goods such as bottles and strollers), hospital or medical industry (e.g., hospital beds, medical equipment), aviation industry (e.g., airplane parts), and building and construction industry (e.g., power tools), among others.

Although only a few embodiments have been disclosed in detail above, other modifications are possible. All such modifications are intended to be encompassed within the following claims.

What is claimed is:

1. A thermoplastic elastomer (TPE) material comprising:
a styrene-ethylene-butadiene-styrene block copolymer rubber;
a Food and Drug Administration (FDA) grade mineral oil; and
a heat and UV stabilizer package.

2. The thermoplastic elastomer (TPE) material of claim 1 wherein the styrene-ethylene-butadiene-styrene block copolymer rubber is present at 10-50%.

3. The thermoplastic elastomer (TPE) material of claim 2 wherein the FDA grade mineral oil is present at 30-70%.

4. The thermoplastic elastomer (TPE) material of claim 1 further comprising an antimicrobial additive that prevents material degradation from exposure to bacteria, fungi, and algae.

5. A method comprising:
extruding a thermoplastic elastomer (TPE) material into a thin film roll with a thickness from 0.003″ to 0.010″, the TPE material comprising a styrene-ethylene-butadiene-styrene block copolymer rubber, a Food and Drug Administration (FDA) grade mineral oil, and a heat and UV stabilizer package.

6. The method of claim 5 wherein the thin film roll has a thickness of greater than 0.010″.

7. The method of claim 6 wherein the thin film roll has a static coefficient of friction of greater than at 1.5.

8. The method of claim 7 wherein the thin film roll has a dynamic coefficient of friction of at least 1.2.

9. The method of claim 8 wherein the thin film roll has a durometer reading of <15 shA.

10. The method of claim 5 wherein the styrene-ethylene-butadiene-styrene block copolymer rubber is present at 10-50%.

11. The method of claim 5 wherein the FDA grade mineral oil is present at 30-70%.

12. The method of claim 5 wherein the TPE material further comprises an antimicrobial additive that prevents material degradation from exposure to bacteria, fungi, and algae.
